(19) United States
(12) Patent Application Publication
(10) Pub. No.: US 2023/0355270 A1
(43) Pub. Date: Nov. 9, 2023

(54) CANNULA AND OBTURATOR SYSTEM FOR MINIMALLY INVASIVE SURGERY

(71) Applicant: Maruho Medical, Marietta, GA (US)

(72) Inventors: Brendan D. Thies, Woodstock, GA (US); Justin C. Sluder, Dallas, GA (US); Brett Andrew Snyder, Beno, NV (US); John L. Minck, JR., Reno, NV (US)

(73) Assignee: Maruho Medical, Marietta, GA (US)

(21) Appl. No.: 17/876,639

(22) Filed: Jul. 29, 2022

Related U.S. Application Data
(60) Provisional application No. 63/231,833, filed on Aug. 11, 2021.
(60) Provisional application No. 63/338,920, filed on May 6, 2022.

(51) Int. Cl. A61B 17/34

(52) U.S. Cl. CPC A61B 17/3423; A61B 17/3431

(57) ABSTRACT

A cannula for providing a pathway for surgical instruments in a minimally invasive procedure having a flexible body portion having a flexible flange extending radially outwardly from the distal region of the flexible body portion. A first seal is positioned within the body portion to prevent egress of fluids from the body of the patient. A rigid ring is positioned at the distal region of the body portion, the ring having an opening dimensioned to receive at least a distal portion of an obturator tip therethrough and a surface surrounding the opening in the ring onto which at least a portion of the obturator contacts to prevent further passage of the obturator.

This application claims priority to provisional application 63/231,833 filed on Aug. 11, 2021, and 63/338,920, filed on May 6, 2022. The entire contents of each of these applications are incorporated herein by reference.

### BACKGROUND

### Technical Field

This application relates to a system and method for accessing the body for minimally invasive surgery, and more particularly to a cannula, insertable with an obturator, for providing a pathway for surgical instruments.

### Background of Related Art

The advantages of minimally invasive surgery over open surgery are well known. These advantages include reduced trauma, lower risk of infection, shorter hospital stays, faster patient recovery time, reduced costs, etc. In minimally invasive surgery, such as endoscopy and arthroscopy, a cannula is inserted through the skin and advanced to the target site. The cannula has a lumen forming a passageway (pathway) for insertion of surgical instruments therethrough to perform the surgical procedure at the target site.

In the design of cannulas, a difficult balance needs to be made between ensuring the cannula has sufficient flexibility during use while ensuring it also has sufficient rigidity for insertion. Thus, while adding stiffer material to the cannula to aid insertion, it reduces the flexibility. It would be beneficial to provide a cannula that optimizes the balance of these two competing factors of rigidity/stiffness and flexibility.

Additionally, the cannula needs to prevent fluid from escaping from the body prior to and during insertion of the instruments therethrough as well as when the instruments remain in the cannula during the surgical procedure. It would be beneficial to provide such fluid blocking structure which does not increase the difficultly of insertion of instrumentation through the cannula.

Several prior art cannulas are introduced with obturators. The need exists to improve such obturator assisted insertion.

Several cannulas are disclosed in the prior art such as by way of example the hybrid cannula of U.S. Pat. No. 9,119,663, the arthroscopic cannula of U.S. Pat. No. 9,675,379, the portal device of U.S. Pat. No. 8,128,601, the arthroscopic cannula of U.S. Publication No. 2021/0128142, the portal device of U.S. Pat. No. 8,128,601 and the cannula of U.S. publication 2019/0239922. Each of these devices/cannulas suffers from one or more deficiencies and fails to address the aforementioned insertion challenges.

The need therefore exists for an easy to manufacture cannula that provides for ease of insertion, achieves an optimal balance of flexibility and rigidity and provides an effective seal to prevent egress of body fluids through the cannula.

### SUMMARY OF THE INVENTION

The present invention overcomes the problems and deficiencies of the prior art.

The present invention provides in one aspect a cannula providing a pathway through the cannula lumen to the target site for various surgical instruments. The present invention provides in another aspect an obturator and cannula system to ease penetration through the patient's skin and advancement to the surgical site followed by removal of the obturator to provide a pathway for instrumentation to the target tissue through the cannula lumen. These components and system are discussed in more detail below.

The devices, systems and methods of the present invention provide an easy to manufacture cannula that provides one or more of a) ease of insertion, b) prevention of slipping out of the incision and c) effective sealing to prevent egress of body fluids through the cannula prior to and during insertion/withdrawal of surgical instruments through the cannula. These advantages will be appreciated by the detailed description of preferred embodiments below. The devices, systems and methods of the present invention can be used in various endoscopic and arthroscopic procedures, including for example, shoulder, hip, knee, elbow, etc.

In accordance with one aspect of the present invention, a cannula for providing a pathway for surgical instruments in a minimally invasive procedure is provided. The cannula comprises a flexible body portion having a proximal region, a distal region, and an intermediate region extending between the proximal region and distal region. The flexible body portion further has a flange, a longitudinal axis, a lumen extending along the longitudinal axis from the proximal region to the distal region, a proximal opening at the proximal region and a distal opening at the distal region. The flange extends radially outwardly from the distal region of the flexible body portion and is flexible for insertion through an incision in a body of a patient. A first seal is positioned within the body portion to prevent egress of fluids from the body of the patient. A rigid ring is positioned at the distal region of the body portion, the ring having an opening dimensioned to receive at least a distal portion of an obturator tip therethrough and a surface surrounding the opening in the ring onto which at least a portion of the obturator e.g., a more proximal portion of the obturator tip, contacts to prevent further passage of the obturator.

In some embodiments, the flange has an asymmetric shape. In some embodiments, the flange is foldable back during insertion and returns to a transverse position when placed within the body of the patient. In some embodiments, the flange is non circular in shape to aid twisting when the cannula is manually rotated by a clinician during insertion.

In some embodiments, the ring has a width less than a width of the flange. The ring in some embodiments is embedded in the flange.

In some embodiments, the seal is positioned at a proximal portion of the cannula; in other embodiments, the seal is positioned at a mid portion of the cannula and can be molded as one piece with the flexible body portion.

The cannula can include, in some embodiments, a proximal end cap positioned over the seal such that the seal is sandwiched between the body portion and the end cap.

In some embodiments, the present invention provides the cannula with one or more of the foregoing features in combination with an obturator, wherein a distal portion of the obturator tip has a diameter less than a diameter of the proximal portion of the obturator tip such that the obturator applies a distal force against the ring during insertion. That is, in such embodiments, the obturator tip extends through the ring opening until a larger diameter portion of the tip abuts the surface of the ring around the opening which thereby provides a stop for obturator insertion. At this point, the obturator can apply a force against the ring to aid insertion.

In accordance with another aspect of the present invention, a method of inserting a cannula is provided comprising a) inserting an obturator into a proximal opening in the cannula, the cannula having a body portion, a lumen, a proximal opening, a distal flange having a distal opening, and a distal ring at a distal portion of the cannula; b) further inserting the obturator through the lumen such that at least a portion of a distalmost tip at the distal region of the obturator exits the distal opening, the ring forming a stop to limit insertion of the obturator; and c) inserting the obturator and cannula together through tissue with the distal region of the obturator contacting a surface of the distal ring and applying a distal force to the ring during insertion.

In some embodiments, the flange has an asymmetric shape. In some embodiments, a width of the flange is greater than a width of the ring. In some embodiments, an opening in the ring has a diameter less than a diameter of the lumen of the cannula.

In some embodiments, during the step of inserting the obturator and cannula, the flange is bent back proximally and after insertion into a body cavity, the flange returns to a transverse position.

In some embodiments, the distal tip of the obturator has a distal region having a diameter less than a diameter of an opening in the distal ring and a more proximal region of the distal tip has a diameter greater than the diameter of the opening in the distal ring.

### BRIEF DESCRIPTION OF THE DRAWINGS

Preferred embodiment(s) of the present disclosure are described herein with reference to the drawings wherein:

FIG. 1 is a perspective view of the obturator and cannula in accordance with an embodiment of the present invention, the obturator shown fully inserted into the cannula;

FIG. 2 is a perspective view of the obturator and an exploded view of the cannula of FIG. 1;

FIG. 3A is a cross sectional view of the obturator and cannula of FIG. 1;

FIG. 3B is a cross sectional view of the obturator and cannula in accordance with another embodiment of the cannula of the present invention;

FIG. 4 is a cross sectional view of the obturator and cannula of FIG. 3A showing the obturator inserted into the cannula;

FIGS. 5A-8 illustrate a method of insertion of the cannula and obturator of FIG. 1 in accordance with an embodiment of the present invention wherein

FIG. 5A shows the obturator prior to insertion into the cannula;

FIG. 5B shows full insertion of the obturator into the cannula wherein the distal tip of the obturator contacts the distal ring of the cannula and the obturator and cannula are positioned against the skin for penetration;

FIG. 6 shows insertion of the obturator and cannula through the tissue wherein the flange of the cannula is folded back and the cannula and obturator are advanced through the tissue;

FIG. 7 shows the cannula and obturator in position in the body cavity;

FIG. 8 shows the obturator being withdrawn from the cannula and the cannula remaining in the body; and

FIG. 9 shows a surgical instrument inserted through the cannula positioned in the body of a patient.

### DETAILED DESCRIPTION OF PREFERRED EMBODIMENTS

The present invention provides a cannula that provides a pathway for surgical instrumentation in endoscopic or arthroscopic surgery. The cannula of the present invention has features that achieve one or more of the following: 1) facilitates ease of insertion into the body; 2) reduces the chances of cannula slippage out of the body; 3) provides an optimal balance of flexibility and rigidity; 4) enhances sealing of the cannula; and 5) facilitates one-handed insertion and manipulation. Features of the cannula that achieve these advantages are described in detail below. It should be understood that the various features (#1 #5) can achieve one or more of the foregoing (#1 #5) and that the cannula need not include all these features but can include any number of features to achieve any number of the foregoing advantages.

The present invention can also include a system that comprises the cannula and an obturator to aid insertion. The obturator is inserted into the lumen of the cannula and inserted together with the cannula as a unit, the obturator bottoming out on a distal ring of the cannula to provide a distal force on the cannula during insertion. This cannula/obturator system is described in more detail below.

Note as used herein, the term “distal” refers to the region or portion further from the user and the term “proximal” refers to the region or portion closer to the user. As used herein, the term “substantially” or “approximately” or “generally” or “about” means+25% of a given value or measurement or feature.

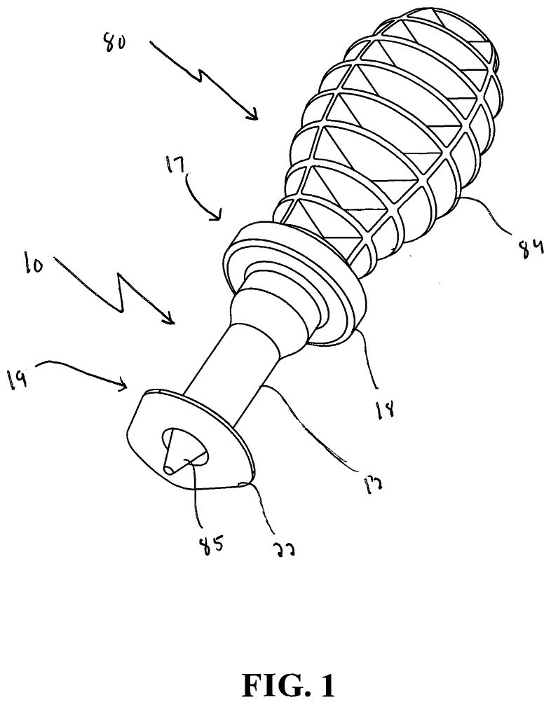

Referring now to the drawings and particular embodiments of the present disclosure, wherein like reference numerals identify similar structural features of the devices disclosed herein, the cannula of the present invention will be discussed first, followed by a discussion of the obturator and the method of use. With initial reference to FIGS. 1, 2 and 3A, the cannula is designated generally by reference numeral 10 and includes a flexible body 12 (also referred to herein as the body portion 12), a distal ring 14, a seal mechanism 16 and a proximal end cap 18.

Body 12 has a proximal portion 17, a distal portion 19 and an intermediate portion extending between the proximal and distal portions 17, 19. In the illustrated embodiment, the flexible body 12 has a uniform diameter along its length, although alternatively it could have a taper in a distal direction so the diameter is reduced in a distal direction to aid insertion through tissue. The taper can also assist sealing the cannula 10 against the surgical incision and help prevent cannula pistoning. Thus, in such embodiments, the distal end of the cannula tapers from the proximal end of the neck to the distal end of the neck to prevent the cannula from pistoning into the body as well as helping to get a good seal between the outside of the cannula neck and the skin incision. It should also be appreciated that in alternate embodiments, the taper could be in a proximal direction of the flexible body 12. It is also contemplated that the outer diameter of the flexible body 12 can change via a stepped construction rather than a taper.

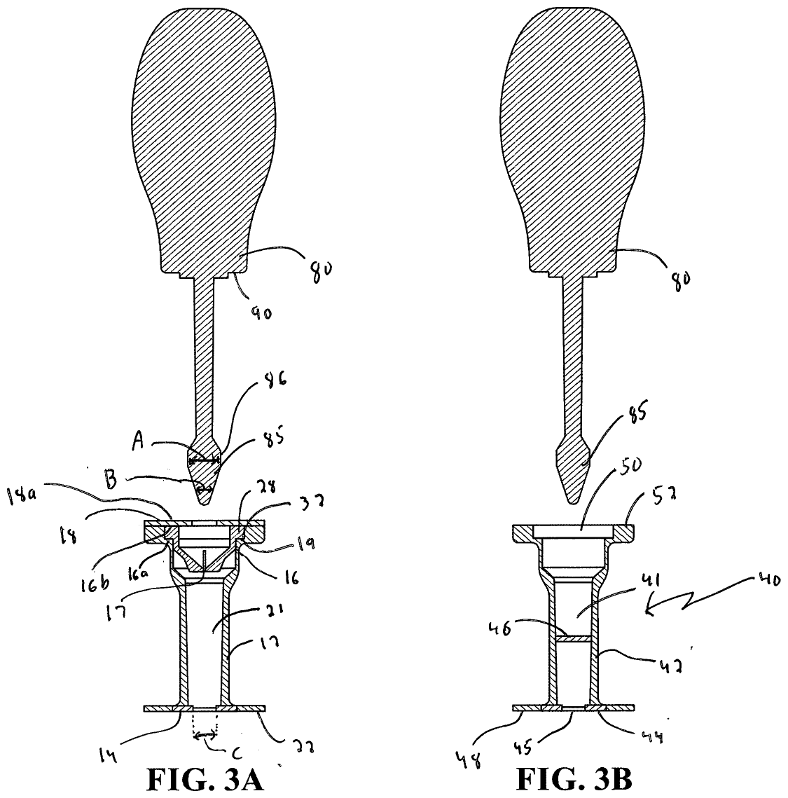

The flexible body 12 also has an enlarged diameter region 13 at the proximal portion which receives seal 16. Flexible body 12 has a lumen 21 (see FIG. 3A) extending therethrough (along the longitudinal axis) with a proximal opening for entry of surgical instrumentation and a distal opening 24 for exit of surgical instrumentation. The lumen 21 is also dimensioned and configured to receive obturator (dilator) 80 therethrough for insertion of the cannula 10 as discussed below. The flexible body 12 can be formed of various polymeric materials such as TPE (thermoplastic elastomer) or silicone by way of example. As shown, the flexible body 12 is preferably circular in cross-section, although other cross-sectional configurations are contemplated, e.g., oval.

The outer surface of the wall of the flexible body 12, along the intermediate region (portion) is preferably smooth, however, in alternate embodiments, a plurality of radially extending tissue engagement features or tissue capture members can be provided as in copending provisional application serial no. 63/231,833, filed Aug. 11, 2021 (hereinafter the '833 application), the entire contents of which are incorporated herein by reference. The engagement members as described in the '833 application are configured and angled to limit the chances of the cannula retracting or pistoning during use, e.g., during insertion and withdrawal of instrumentation therethrough, while not adversely affecting insertion of the cannula through tissue into the body cavity, i.e., without increasing difficulty of insertion which can cause trauma to tissue. The tissue engagement members can also help insertion as the cannula is rotated into tissue.

The distal opening 24 of flexible body 12 could have projections or tabs similar to tabs 44 described in the '833 application which project inwardly toward the longitudinal axis of the flexible body 12 to engage slots in the obturator.

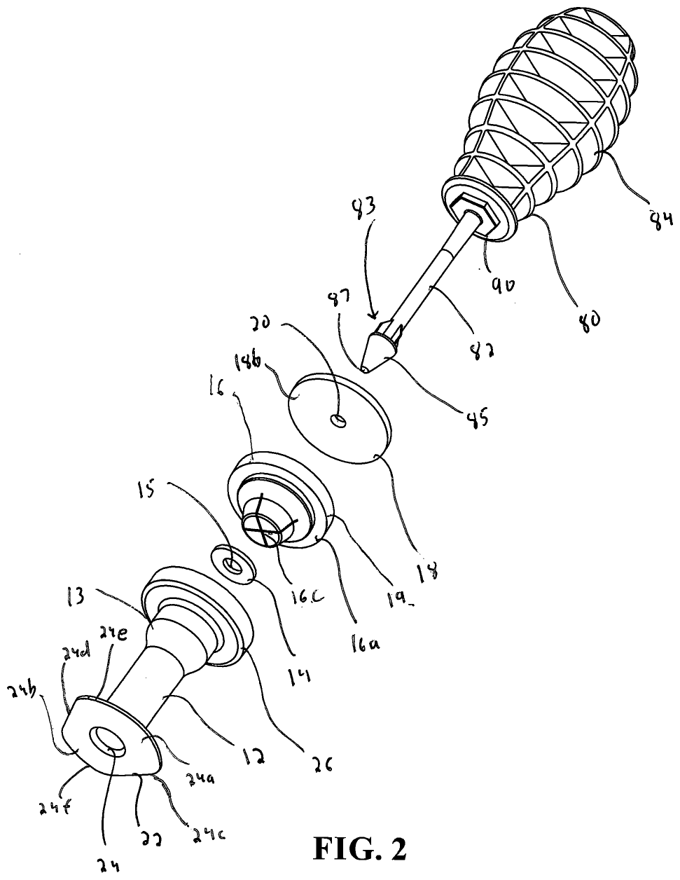

Flexible body 12 has a flange 22 at a distal end extending radially outwardly thereby increasing the diameter of the flexible body portion 12. The flange 22 preferably has a non-circular asymmetric shape as shown. In the illustrated embodiment, the asymmetric flange has a wider region 24a and a narrower region 24b. Some of the walls can be substantially straight and others curved; alternatively, all of the walls can have a curvature. In the embodiment of FIG. 2, the two side walls 24e, 24d are generally straight, end wall 24e has a slight curve and end wall 24f is arcuate. Stated another way, side walls 24c 24d angle outwardly from end wall 24f towards end wall 24e. Note the walls are referred to as “side” and “end” walls for convenience to explain the asymmetric configuration. Other asymmetric shapes than that shown are also contemplated. The asymmetric shapes aids insertion as the flange 22 is foldable as it passes through tissue. The asymmetric feature also provides an alignment or orientation feature of the cannula to indicate the position of the cannula. The alignment can be via association of the shapes of the flange or alignment features or markings can be placed on the flange. The asymmetry also reduces the amount of material of the flange which increases its flexibility.

The flange 22 surrounds the distal opening 24 and has a continuous outer periphery. Alternatively, the flange can include a plurality of spaced apart fingers as in fingers 36 of the '833 application.

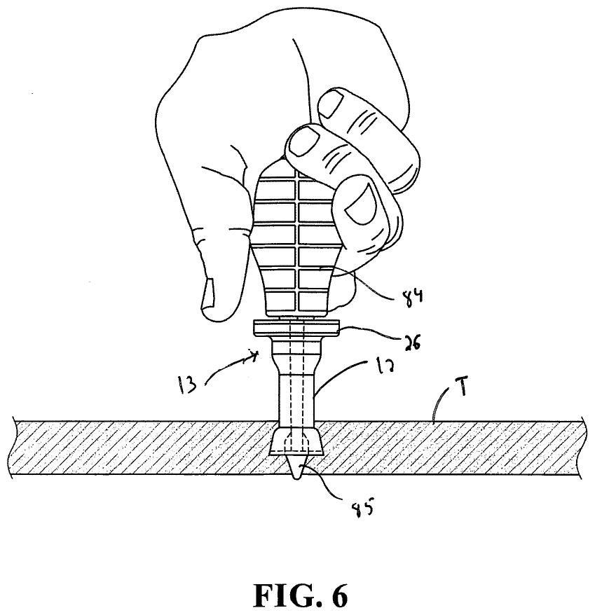

The flange 12 performs two functions. One function is to provide a stop for retraction of the cannula 10 once the cannula 10 is positioned in the body cavity. A second function is to aid insertion as the flange 22 bends back proximally during insertion as the cannula 10 is pushed distally and/or rotated during insertion. This proximal bending of the flange during insertion through tissue is illustrated in FIG. 6 and discussed below in conjunction with the discussion of the method of use/insertion.

Ring 14 is positioned at a distal region of the cannula 12, preferably at a distalmost end. In some embodiments it can be embedded in, e.g., overmolded with the flange 22, as in the illustrated embodiment of FIG. 3A. In other embodiments, the ring 14 can be positioned proximal of the flange 22, e.g., attached to a proximal surface of the flange 22. In any case, the ring 22 provides a solid rigid part (rigid component) within the flexible body 12 to enhance pushablility of the cannula 10 for insertion.

Ring 14 has an opening 15 aligned with lumen 21 of body 12 and with opening 24 of flange 22. The ring 14 preferably has a width, e.g., diameter, less than a width of the flange 22. In this manner, as shown in FIG. 3A, the ring 14 does not adversely affect the flexibility of the flange 22 as it is seated mostly inwardly of the outer surfaces (and inward of the end/side walls 24c-24f) of flange 22. The opening 15 of ring 14 has a diameter less than a diameter of the lumen 21 and less than a diameter of a portion of the nose of the obturator 80. In this way, the ring 14 can provide a stop for the obturator 80 as described herein. This can be appreciated by reference to FIG. 3A wherein the opening 15 in ring 14 has a dimension, e.g., diameter, “C” which is greater than dimension, e.g., diameter or width, “B” at a distal region of tip 85 of obturator 80 to allow passage therethrough but dimension “E” is smaller than dimension, e.g., diameter or width, “A” at a proximal region of tip 85 to prevent further passage of obturator 85, thus enabling the ring 14 to provide a distal stop for obturator passage through the cannula 10. Note the ring opening diameter and the obturator diameter can be dimensioned so a different part of the obturator tip, or a different part of the obturator, comes into contact with the surface around the opening ring to limit distal advancement of the obturator within the cannula.

With reference to FIG. 3A, body 12 has an internal shoulder 32 at the proximal end which the rim 19 of the seal 16 abuts. That is, the undersurface 16a of rim 19 of seal 16 abuts the upper (proximal) surface of shoulder 32. The internal shoulder 32 is formed in the large diameter region 13 of the flexible body 12. End cap 18 sits atop the seal 16 and upper surface of the body 12 such that seal 16 is sandwiched between the end cap 18 and shoulder 32. End cap 18 has an opening 20 aligned with the proximal opening in body 12 and aligned with the lumen 21. The outer (proximal) surface of end cap 18 is designated by reference numeral 18a and the inner (distal) surface, which contacts the proximal surface of body 12 and proximal surface 16b of the seal 16, is designated by reference numeral 18b. In the illustrated embodiment, the seal 16 is seated entirely within the enlarged region 13 of the body 12 and has a diameter less than the diameter of proximal rim 26 of body 12.

The seal 16 provides a cannula seal which seals the cannula from egress of fluid in the absence of an instrument inserted or positioned in the lumen 21 of the cannula. The cannula seal (also referred to herein as the access valve) 16 can be in the form of a slit valve having slit 16c for passage of instruments. The seal 16 provides an instrument seal which seals the cannula from egress of fluid when an instrument, e.g., obturator, grasper, cutting instrument, etc. is inserted through or positioned within the lumen of the cannula 10. Other types of valves/seals are also contemplated. In some embodiments, seal 16 can remain closed when no instrument is passed therethrough. The valve seals the cannula and enables instruments to be inserted through the slit 16c. The seal 16 can also include an elastic membrane with a slot or opening to open when an instrument is inserted therethrough and seals around the instrument. The proximal surface 16b of seal 16, as explained above, abuts the distal surface 18b of end cap 18. The distal surface 16a of seal 16 abuts the proximal surface of shoulder 32.

In the alternate embodiment of FIG. 3B. instead of the separate seal component 16 attached to the body 12, the seal is molded with the flexible body. More specifically, cannula 40 has a proximal opening 50, a distal opening 45, and a lumen 41 extending along the longitudinal axis from the proximal to the distal opening. The cannula has a flange 48 and ring 44. The flange 48 is identical to flange 22 of FIG. 3A and ring 45 is identical to ring 14 of FIG. 3A. Therefore, for brevity, the description of the flange and the ring is not repeated herein it being understood that the discussion of the features of the flange 22 and the ring 14 of FIGS. 2 and 3A, and their function and alternative designs, are fully applicable to flange 48 and ring 44 of FIG. 3B. Note the seal 46 is shown in a mid-portion of cannula 40, although it is also contemplated that the seal could be formed in a more proximal or more distal position than that shown. An additional seal such as seal 16 of FIG. 2 can be provided in cannula 40.

For manufacture of the cannula 10 of FIG. 3A, the flexible body portion 12 can be formed by injection molding. Seal 16 can then be placed in the rigid body 14 with the distal surface 16a of seal 16 engaging shoulder 32 of body 12 (see FIG. 3A). The end cap 18 is then fastened to body 12 by ultrasonic welding, glue or other methods. The seal (valve) 16 is thereby sandwiched between the end cap 18 and the internal shoulder 32 of body 12 to retain the seal 16 within the proximal rim 26 of enlarged region 13 of body 12.

The cannula 10 (and cannula 40 of FIG. 3B) is inserted with the aid of obturator 80 removably positioned therein. With reference to FIG. 2, obturator 80 (also referred to herein as a dilator) has a shaft portion 82, a distal portion 83 and a proximal portion 84 forming a grip portion for the user/clinician. The obturator 80 terminates at distal portion 83 in a distal tip 85 with a distalmost tip 87. The distal tip 85 in the illustrated embodiment is conical in shape, although other shaped tips to aid penetration through body tissue are also contemplated. The tapering of the tip 85 is explained above with respect to the demarcated dimensions of FIG. 3A.

The obturator 80 can have cannula receiving L-shaped slots for engagement with the obturator engaging projections (tabs) of the flexible body portion 12 of cannula 10 as disclosed in the '833 application to lock (interlock) the obturator 80 with the cannula 10 via rotation as described in detail therein. This interlocking aids one handed insertion. Note the obturator 80 as disclosed in the '833 application can be rotated with respect to the cannula for the interlocking, or alternatively, the cannula can be rotated with respect to the obturator 80 or both the obturator 80 and cannula 10 can be rotated in opposing directions to achieve the disclosed tab-slot engagement/interlocking.

The obturator 80 can include a shoulder 90 to engage the proximal surface 18a of end cap 18 or in the embodiment of FIG. 3B engage proximal flange 52 of cannula 50. This can provide a limit to distal insertion of the obturator 80 into the body portion 12 of cannula 10. However, the conical tip engagement with ring 14 can instead be the sole feature limiting obturator insertion, or the obturator 80 and/or the cannula 12 can be dimensioned, e.g., have a length, such that limited penetration of the conical tip and ring coincides with the obturator shoulder 90 contacting the end cap outer (proximal) surface 18a (or flange 52 in the embodiment of FIG. 3B). The shaft (body) portion 82 of obturator 80 can be tapered or alternatively of uniform outer diameter along its length as shown. Note the body portion 82 of obturator 80 is preferably solid, although in alternate embodiments it can have a lumen extending therein. The obturator 80 is composed of a rigid material which resists flexing to provide sufficient force during insertion.

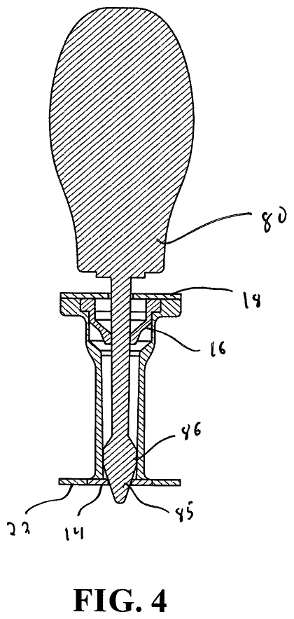

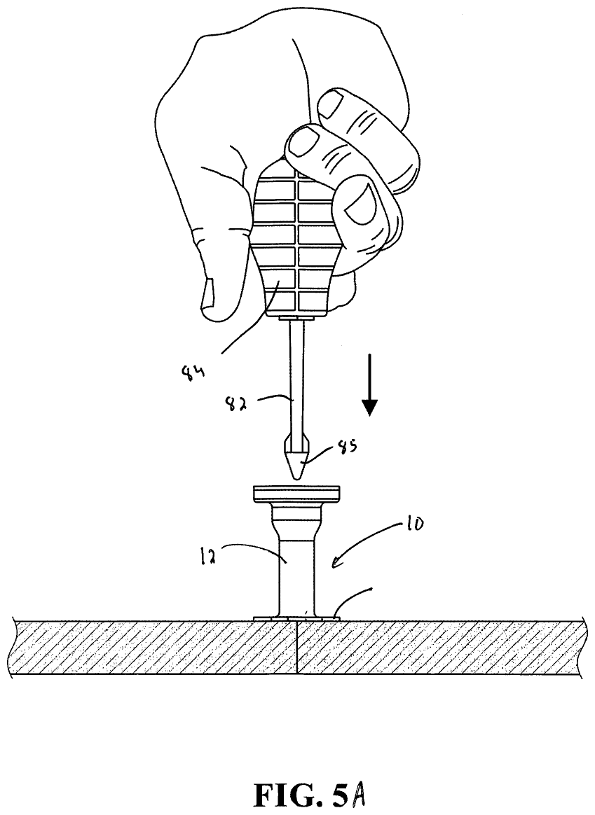

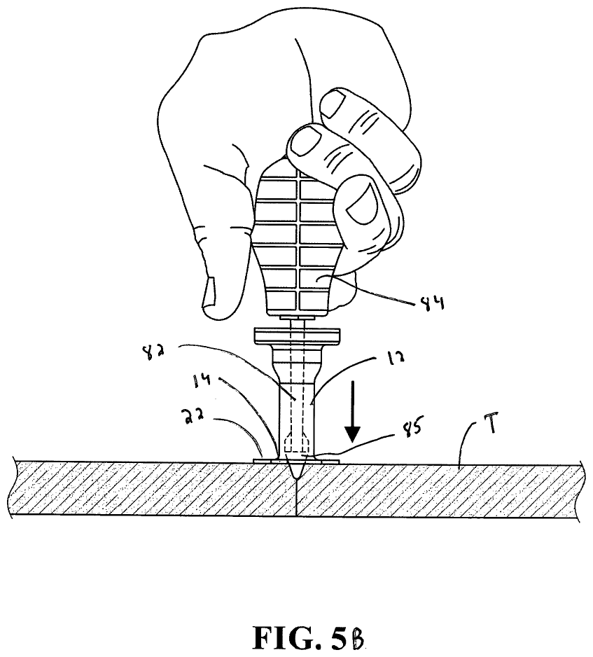

Turning now to the method of use of the system of the present invention, and with reference to FIGS. 5A-9, in the first step, the obturator 80 is inserted into the cannula 10 through seal 16 and through lumen 21 of body 12 so that the conical tip 85 extends beyond the flange 22 of flexible body portion 14 as shown in FIG. 5B. (This is also shown in the cross-sectional view of FIG. 4). Thus, the tip 85 is exposed to aid insertion. (Note cannula 40 of FIG. 3B would be inserted in the same manner as cannula 10)

Once fully inserted into the cannula 10, the obturator tip 85 contacts the rigid ring 14 (due to the dimension of the ring opening) which prevents its further insertion. The obturator 80 is then pushed distally, applying a distal force on the ring 14 which thereby applies a distal force to the cannula 10 to advance it through tissue. The obturator 80 in some embodiments can also be rotated during insertion. Note during insertion the clinician not only grips the grip portion 84 of obturator as shown in FIG. 6, but can also grip the enlarged region 13, e.g., enlarged rim 26 of cannula 12 to thereby allow for control, e.g., insertion, with one hand. That is, in this one handed user, the user's palm can grip the obturator 80 at the gripping portion 84 and the user's finger tips, e.g., the thumb and little finger, can grip the enlarged rim 26 of the cannula 10.

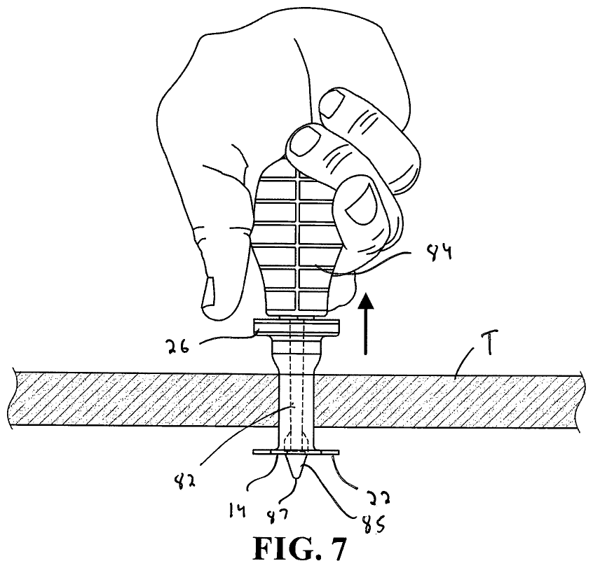

As the cannula 10 is being inserted through the incision and through tissue T, the flange 22 bends backward (proximally) from the transverse position of FIG. 5A to the position of FIG. 6. Once the cannula 10 is advanced into the body cavity, the flange 22 returns to its transverse position. The insertion position wherein the cannula 10, with the assistance of obturator 80, is positioned in the body cavity adjacent the target site, and the flange 22 returns to its transverse position, is shown in FIG. 7.

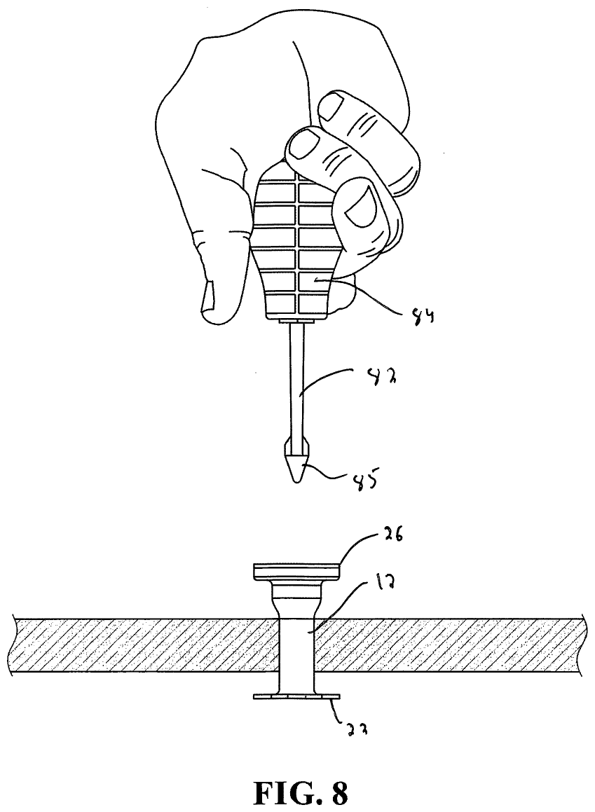

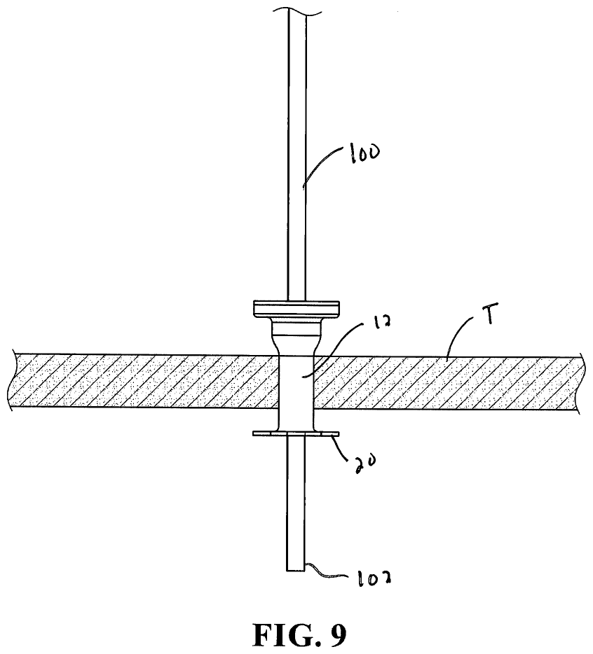

Once the cannula 10 is positioned adjacent the target site, the obturator 80 is removed from the lumen 21 of the body portion 12 of cannula 10 in the direction of the arrow of FIG. 7 until it is fully removed as shown in FIG. 8, leaving the cannula 10 in the body cavity. Instruments such as instrument 100 can then be inserted through the seal 16 and through the lumen 21 with the distal end 102 exiting past the distal end of the body portion 12 for treating the target site (see FIG. 9). Note if the tissue engagement members are provided, these will help retain the flexible body portion 12 in position. Further note that seal 16 seals around the obturator 80 and subsequent instruments as described above. After the surgical procedure, the cannula 10 is retracted out of the tissue T for removal from the patient's body.

It should be appreciated that in alternate methods of use, the cannula 10 can be inserted without an obturator.

Although the apparatus and methods of the subject invention have been described with respect to preferred embodiments, those skilled in the art will readily appreciate that changes and modifications may be made thereto without departing from the spirit and scope of the present invention as defined by the appended claims. Persons skilled in the art will understand that the various embodiments of the disclosure described herein and shown in the accompanying figures constitute non-limiting examples, and that additional components and features may be added to any of the embodiments discussed herein without departing from the scope of the present invention.

It will be understood by those skilled in the art that the above particular embodiments are shown and described by way of illustration only. The principles and the features of the present disclosure may be employed in various and numerous embodiments thereof without departing from the scope and spirit of the disclosure as claimed. The above-described embodiments illustrate the scope of the disclosure but do not restrict the scope of the disclosure.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the present invention, terms such as “approximately,” “about”, “generally,” “substantially,” and the like should be understood to allow for variations in any numerical range or concept with which they are associated. It is intended that the use of terms such as “approximately”, “about”, “substantially”, and “generally” should be understood to encompass variations on the order of 25%, or to allow for manufacturing tolerances and/or deviations in design.

The recitation of numerical ranges by endpoints includes all numbers within the range. Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A cannula for providing a pathway for surgical instruments in a minimally invasive procedure, the cannula comprising:
a) a flexible body portion having a proximal region, a distal region, and an intermediate region extending between the proximal region and distal region, the flexible body portion further having a flange, a longitudinal axis, a lumen extending along the longitudinal axis from the proximal region to the distal region, a proximal opening at the proximal region and a distal opening at the distal region, the flange extending radially outwardly from the distal region of the flexible body portion and being flexible for insertion through an incision in a body of a patient;
b) a first seal positioned within the body portion to prevent egress of fluids from the body of the patient; and
c) a rigid ring positioned at the distal region of the body portion, the ring having an opening dimensioned to receive at least a distal portion of an obturator tip therethrough and a surface surrounding the opening in the ring onto which at least a portion of the obturator contacts to prevent further passage of the obturator.

2. The cannula of claim 1, wherein the flange has an asymmetric shape.

3. The cannula of claim 2, wherein the flange is foldable back during insertion and returns to a transverse position with respect to the longitudinal axis when placed within the body of the patient.

4. The cannula of claim 1, wherein a proximal portion of the obturator tip contacts the surface surrounding the opening in the ring.

5. The cannula of claim 1, wherein the ring has a width less than a width of the flange.

6. The cannula of claim 1, wherein the seal is positioned at a proximal portion of the cannula.

7. The cannula of claim 6, wherein the seal is positioned at a mid portion of the cannula.

8. The cannula of claim 7, wherein the seal is molded as one piece with the body portion.

9. The cannula of claim 1, wherein the ring is embedded in the flange.

10. The cannula of claim 6, further comprising a proximal end cap positioned over the seal, and the seal is sandwiched between the body portion and the end cap.

11. The cannula of claim 1, in combination with an obturator, wherein a distal portion of the obturator tip has a diameter less than a diameter of the proximal portion of the obturator tip and the diameter of the proximal portion of the obturator tip is larger than the opening in the ring such that the obturator applies a distal force against the ring during insertion.

12. The cannula of claim 11, wherein the obturator tip is substantially conical.

13. The cannula of claim 1, further comprising an alignment feature on the flange to provide an orientation indicator to a user.

14. A method of inserting a cannula comprising:
a) inserting an obturator into a proximal opening in the cannula, the cannula having a body portion, a lumen, a proximal opening, a distal flange having a distal opening, and a distal ring at a distal portion of the cannula;
b) subsequent to step (a), further inserting the obturator through the lumen of the cannula such that at least a portion of a distal tip of the obturator exits the distal opening, the ring forming a stop to limit insertion of the obturator; and
c) inserting the obturator and cannula together through tissue with a distal portion of the obturator contacting the ring and applying a distal force to the ring during insertion.

15. The method of claim 14, wherein a portion of the distal tip of the obturator contacts the ring adjacent the opening in the ring.

16. The method of claim 14, wherein the flange has an asymmetric shape and a width of the flange is greater than a width of the ring.

17. The method of claim 14, wherein the flange has an asymmetric shape.

18. The method of claim 14, wherein during the step of inserting the obturator and cannula, the flange is bent back proximally and after insertion into a body cavity, the flange returns to a transverse position.

19. The method of claim 14, wherein an opening in the distal ring has a diameter less than a diameter of the lumen of the cannula.

20. The method of claim 14, wherein the distal tip of the obturator has a distal region having a diameter less than a diameter of an opening in the distal ring and a more proximal region of the distal tip has a diameter greater than the diameter of the opening in the distal ring.
